(19) United States
(12) Patent Application Publication
(10) Pub. No.: US 2020/0246193 A1
(43) Pub. Date: Aug. 6, 2020

(54) CONFORMABLE ADHESIVE BANDAGES

(71) Applicant: Tru-Colour Products, LLC, Warrenville, IL (US)

(72) Inventor: Raymond K WURAPA, Blacklick, OH (US)

(21) Appl. No.: 16/774,803

(22) Filed: Jan. 28, 2020

Related U.S. Application Data
(60) Provisional application No. 62/800,886, filed on Feb. 4, 2019.

(51) Int. Cl. A61F 13/02

(52) U.S. Cl. CPC A61F 13/025; A61F 2013/00544; A61F 13/0206; A61F 13/0259

(57) ABSTRACT

A conformable adhesive bandage is disclosed including a central body, a plurality of bandage limbs including an adhesive coating disposed on a first side of the conformable adhesive bandage, and a plurality of flexion sites, each of the plurality of flexion sites inflecting inward into the central body or the plurality of bandage limbs. In one embodiment, the plurality of flexion sites is disposed at a plurality of vertices along the central body between adjacent bandage limbs of the plurality of bandage limbs, the plurality of flexion sites inflecting radially inward at the plurality of vertices into the central body. In another embodiment, is the plurality of flexion sites includes a plurality of central body flexion sites disposed at a plurality of vertices along the central body between adjacent bandage limbs, and a plurality of bandage limb flexion sites disposed along at least one of the plurality of bandage limbs.

### RELATED APPLICATIONS

This application claims the benefit of and priority to U.S. Prov. App. No. 62/800,886, filed Feb. 4, 2019, which is hereby incorporated by reference in its entirety.

### FIELD OF THE INVENTION

This application is directed to adhesive bandages. More specifically, this application is directed to adhesive bandages inducing reduced restriction of joint motion relative to non-inventive adhesive bandages.

### BACKGROUND OF THE INVENTION

The challenge of application of adhesive bandages around joints is their ability to remain in place despite all the surrounding skin motion associated with joint movement. Adhesive bandages that are currently used to cover minor wounds around joint areas of upper and lower extremities consist of an adhesive cloth fabric, with absorbent central pad. Most bandages have a simple wide strip design.

When strip bandages are applied around joints, they can significantly restrict skin motion and thus joint motion during activity, and can be uncomfortable, prone to pulling off, and may even impact blood flow to the tissue. Some bandage designs are adapted specifically for application around joints, and consist of a central body with four parallel limbs, resembling an H-type pattern. These H-type bandages have the advantage of allowing greater movement of the skin around joints during activity, compared to strip type bandages. The disadvantage of these H-type designs, however, is that the orientation of the bandage limbs in relation to the body of the bandage cannot be changed. This restricts the degree to which the bandage limbs are able to contour and fit the varied anatomical prominences around the joints of hands and feet.

The skin contours around joints change dynamically during routine daily activities demanding a better fitting adhesive bandage that is more likely to remain in place maintaining protective wound coverage, while not impeding joint motion. This need is further amplified for more active individuals, those with highly physical occupations, and athletes, where the shear forces across the skin around joints are even greater.

There is a need for adhesive bandages with reduced restriction of joint motion relative to non-inventive bandages.

### BRIEF DESCRIPTION OF THE INVENTION

In one exemplary embodiment, a conformable adhesive bandage includes a central body, a plurality of bandage limbs including an adhesive coating disposed on a first side of the conformable adhesive bandage, and a plurality of flexion sites, each of the plurality of flexion sites inflecting inward into the central body or the plurality of bandage limbs.

In another exemplary embodiment, a conformable adhesive bandage includes a central body, a plurality of bandage limbs including an adhesive coating disposed on a first side of the conformable adhesive bandage, and a plurality of flexion sites, the plurality of flexion sites being disposed at a plurality of vertices along the central body between adjacent bandage limbs of the plurality of bandage limbs, the plurality of flexion sites inflecting radially inward at the plurality of vertices into the central body.

In another exemplary embodiment, a conformable adhesive bandage includes a central body, a plurality of bandage limbs including an adhesive coating disposed on a first side of the conformable adhesive bandage, a plurality of central body flexion sites, the plurality of central body flexion sites being disposed at a plurality of vertices along the central body between adjacent bandage limbs of the plurality of bandage limbs, and the plurality of flexion sites inflect radially inward at the plurality of vertices into the central body, and a plurality of bandage limb flexion sites disposed along at least one of the plurality of bandage limbs and inflecting inward into the at least one of the plurality of bandage limbs, the plurality of bandage limb flexion sites being divided into pairs and each bandage limb flexion site of a pair of bandage limb flexion sites being disposed in opposing edges of a bandage limb at an equivalent position along a length of the bandage limb.

### BRIEF DESCRIPTION OF THE DRAWINGS

FIG. 1 is a top view of a conformable adhesive bandage having two bandage limbs, according to an embodiment of the present disclosure.

FIG. 2 is a top view of a conformable adhesive bandage having three bandage limbs, according to an embodiment of the present disclosure.

FIG. 3 is a top view of a conformable adhesive bandage having four bandage limbs in an H-type configuration, according to an embodiment of the present disclosure.

FIG. 4 is a bottom view of the conformable adhesive bandage of FIG. 3, according to an embodiment of the present disclosure.

FIG. 5 is a side view of the conformable adhesive bandage of FIG. 3, according to an embodiment of the present disclosure.

FIG. 6 is an expanded view of a flexion site of the conformable adhesive bandage of FIG. 3 taken within line 6, according to an embodiment of the present disclosure.

FIG. 7 is an expanded view of a flexion site of the conformable adhesive bandage of FIG. 3 taken within line 7, according to an embodiment of the present disclosure.

FIG. 8 is an expanded view of a flexion site of the conformable adhesive bandage of FIG. 3 taken within line 8, according to an embodiment of the present disclosure.

FIG. 9 is an expanded view of a flexion site of the conformable adhesive bandage of FIG. 3 taken within line 9, according to an embodiment of the present disclosure.

FIG. 10 is a top view of a conformable adhesive bandage having four bandage limbs in an asymmetric H-type configuration, according to an embodiment of the present disclosure.

FIG. 11 is a top view of a conformable adhesive bandage having four bandage limbs in an X-type configuration, according to an embodiment of the present disclosure.

FIG. 12 is a top view of a conformable adhesive bandage having five bandage limbs, according to an embodiment of the present disclosure.

FIG. 13 is a top view of a conformable adhesive bandage applied to a hand, according to an embodiment of the present disclosure.

FIG. 14 is a bottom view of the conformable adhesive bandage applied to a hand of FIG. 13, according to an embodiment of the present disclosure.

Wherever possible, the same reference numbers will be used throughout the drawings to represent the same parts.

### DETAILED DESCRIPTION OF THE INVENTION

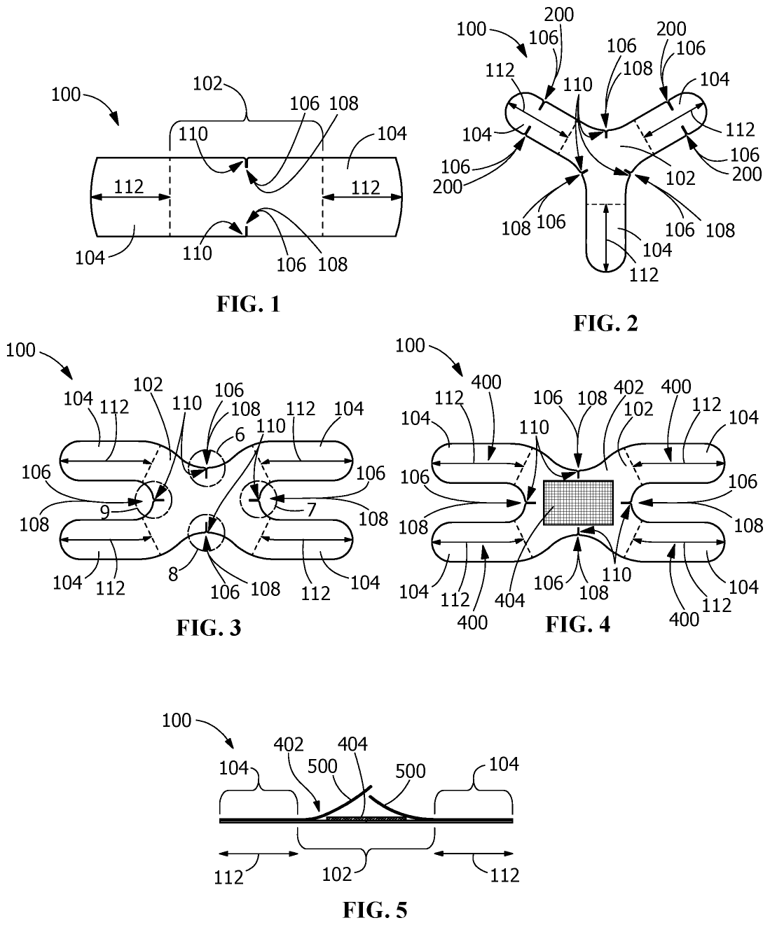

Referring to FIGS. 1-14, in one embodiment, a conformable adhesive bandage 100 includes a central body 102, a plurality of bandage limbs 104, and a plurality of flexion sites 106, each of the plurality of flexion sites 106 inflecting inward into the central body 102 or the plurality of bandage limbs 104. Referring to FIG. 4, conformable adhesive bandage 100 includes an adhesive coating 400 disposed on a first side 402 of the conformable adhesive bandage 100.

The plurality of bandage limbs 104 may include or consist of any suitable number of bandage limbs 104, including, but not limited to, two bandage limbs 104 (FIG. 1), three bandage limbs 104 (FIG. 2), four bandage limbs 104 (FIGS. 3-11, 13, and 14), five bandage limbs 104 (FIG. 12), or more. Each bandage limb 104 may be straight, curved, angled, or any combination thereof. A single conformable adhesive bandage 100 may include identical bandage limb 104 conformations, symmetric bandage limb 104 conformations, asymmetric bandage limb 104 conformations, distinct bandage limb 104 conformations, or any combination thereof.

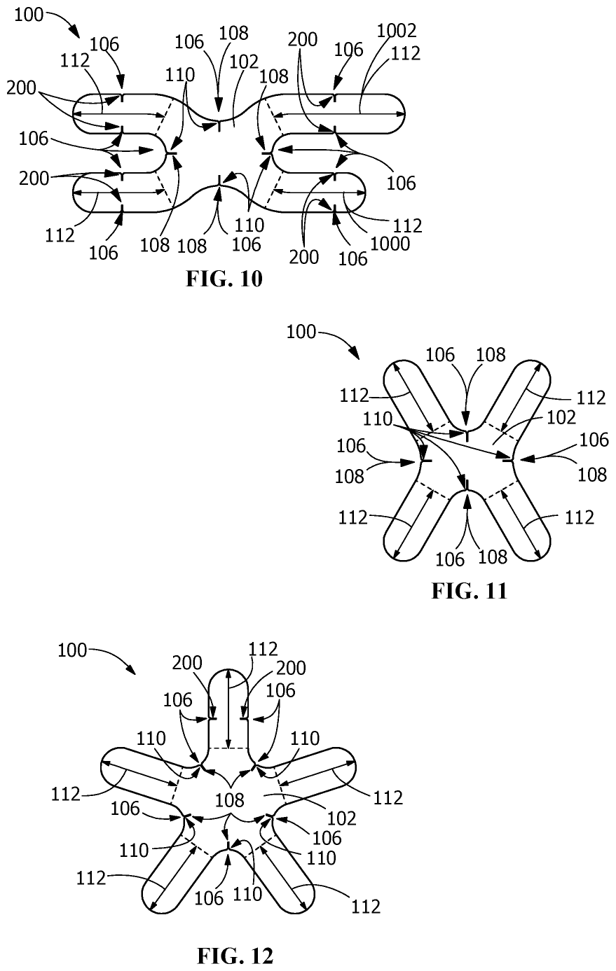

The may be symmetric in orientation or asymmetric in orientation. The plurality of bandage limbs 104 may be equal in length 112 (FIGS. 1-5, 11, and 12) or unequal in length 112 (FIG. 10). Referring to FIG. 10, in one embodiment, at least one of the plurality of bandage limbs 104 includes a first bandage limb length 1000, and at least one of the plurality of bandage limbs includes a second bandage limb length 1002, the second bandage limb length 1002 being longer than the first bandage limb length 1000.

Referring to FIGS. 3 and 11, in one embodiment, wherein the plurality of bandage limbs 104 consists of four bandage limbs 104, the arrangement of the bandage limbs 104 may include an alignment relative to one another of up to 10° from parallel, which is referred to as an H-type configuration (FIG. 3), or an alignment relative to one another in excess of 10° from parallel, which is referred to as an X-type configuration.

Referring to FIG. 4, in one embodiment, the conformable adhesive bandage 100 includes an absorbent pad 404 disposed centrally on the central body 102 on the first side 402 of the conformable adhesive bandage 100. Referring to FIG. 5, in another embodiment, the conformable adhesive bandage 100 includes at least one removable backing 500 disposed on the first side 402 of the conformable adhesive bandage 100.

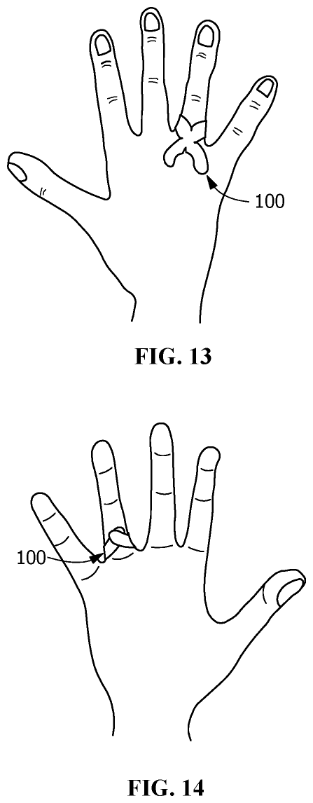

Referring to FIGS. 13 and 14, in one embodiment, the conformable adhesive bandage 100 minimizing restriction of joint motion relative to an otherwise identical comparative bandage lacking the plurality of flexion sites 106. Without being bound by theory, it is believed that the plurality of flexion sites 106 renders the conformable adhesive bandage 100 customizable for dynamic contouring with skin, particularly around joints, by increasing independent adjustment of each of the plurality of bandage limbs 104.

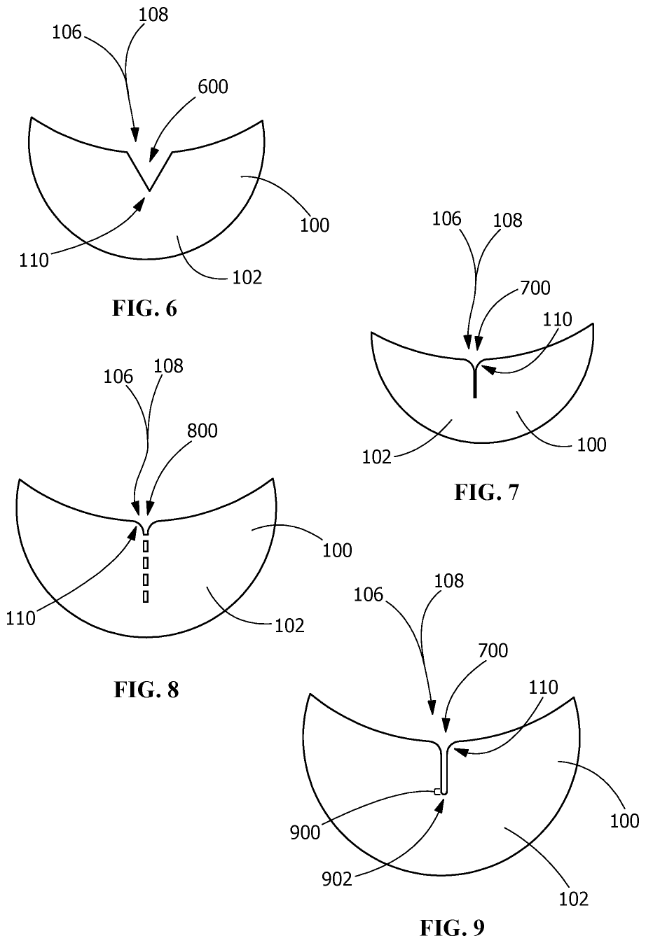

Referring to FIGS. 1-4 and 6-12 each of the plurality of flexion sites 106 increases flexibility of plurality of bandage limbs 104. Referring to FIG. 6, in one embodiment, a flexion site 106 includes an inward notch 600 into the central body 102 or a bandage limb 104. As used herein, a “notch” refers to a v-shaped indentation. Referring to FIG. 7, in another embodiment, a flexion site 106 includes a slit 700 into the central body 102 or a bandage limb 104. As used herein, “slit” refers to a straight incision or incision formed by two curves converging to a point at the apex, such as semicubical parabola. Referring to FIGS. 7 and 8, the slit 700 may be an unperforated slit 700 (FIG. 7) or perforated slit 800 (FIG. 8). Referring to FIG. 9, in yet another embodiment, a flexion site 106 includes a concave curvature 900 into the central body 102 or a bandage limb 104. The concave curvature 900 may be for the entirety of the flexion site 106 or may be limited to an inward apex 902 of the flexion site 106. These embodiments are specifically subject to combinations with one another. Without being bound by theory, it is believe that including a concave curvature 900 at an inward apex 902 of the flexion site 106 reduce or eliminate a stress riser at the flexion site 106 relative to an otherwise identical embodiment lacking the concave curvature 900 at the inward apex 902 of the flexion site 106.

A single conformable adhesive bandage 100 may include any combination of flexion site 106 embodiments, and further, each flexion site 106 may include any combination of the embodiments described herein, such as, but not limited to, a slit 700 which includes a concave curvature 900 at the inward apex 902 thereof, of a notch 600 which includes a concave curvature 900 at the inward apex 902 thereof, or a notch 600 which transitions to a slit 700, or a notch 600 which transitions to a slit 700 which transitions to a concave curvature 900 at the inward apex 902 thereof.

The plurality of flexion sites 106 may include central body flexion sites 108, bandage limb flexions sites 200 or both. Central body flexion sites 108 are disposed at a plurality of vertices 110 along the central body 102 between adjacent bandage limbs 104 of the plurality of bandage limbs 104, and the plurality of central body flexion sites 108 inflect radially inward at the plurality of vertices 110 into the central body 102. Bandage limb flexion sites 200 are disposed along at least one of the plurality of bandage limbs 104 and inflect inward into the at least one of the plurality of bandage limbs 104. In embodiments in which bandage limb flexion sites 200 are present, the bandage limb flexion sites 200 may be disposed along one, several, or all of the bandage limbs 104 of the conformable adhesive bandage 100. Bandage limb flexion sites 200 may be disposed at a singular position along a length 112 of the bandage limb 104 for each bandage limb 104 in which the bandage limb flexion sites 200 are present or at distinct positions along the length 112 of the bandage limb 104 for each bandage limb 104 in which the bandage limb flexion sites 200 are present, or any combination thereof. Bandage limb flexion sites 200 may be disposed on a single edge of a bandage limb 104, along both edges of a bandage limb 104 but at differing positions along the length 112 of the bandage limb 104 (in an alternating arrangement), or in pairs of bandage limb flexion sites 200 disposed in opposing edges of a bandage limb at an equivalent position along a length 112 of the bandage limb 104 (in a paired arrangement). Length 112 refers specifically to a distance measured at a mean width of a bandage limb 104 from the central body 102 to a distal terminus of the bandage limb 104.

While the foregoing specification illustrates and describes exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. It is specifically intended that distinct embodiments described herein or depicted in the drawings hereof may be combined with one another. Therefore, it is intended that the invention not be limited to the particular embodiment disclosed as the best mode contemplated for carrying out this invention, but that the invention will include all embodiments falling within the scope of the appended claims.

What is claimed is:

1. A conformable adhesive bandage, comprising:
a central body;
a plurality of bandage limbs including an adhesive coating disposed on a first side of the conformable adhesive bandage; and
a plurality of flexion sites, each of the plurality of flexion sites inflecting inward into the central body or the plurality of bandage limbs.

2. The conformable adhesive bandage of claim 1, wherein the plurality of bandage limbs includes at least three bandage limbs.

3. The conformable adhesive bandage of claim 1, wherein the plurality of bandage limbs includes at least four bandage limbs.

4. The conformable adhesive bandage of claim 3, wherein the plurality of bandage limbs consists of the four bandage limbs.

5. The conformable adhesive bandage of claim 3, wherein the each of the plurality of bandage limbs is aligned within 10° of parallel to one another, defining an H-type configuration.

6. The conformable adhesive bandage of claim 3, wherein each of the plurality of bandage limbs is aligned in excess of 10° from parallel to one another, defining an X-type configuration.

7. The conformable adhesive bandage of claim 1, wherein the plurality of bandage limbs includes at least five bandage limbs.

8. The conformable adhesive bandage of claim 1, wherein each of plurality of flexion sites includes an inward notch into the central body or the plurality of bandage limbs.

9. The conformable adhesive bandage of claim 1, wherein each of plurality of flexion sites includes a slit into the central body or the plurality of bandage limbs.

10. The conformable adhesive bandage of claim 9, wherein the slit is a perforated slit.

11. The conformable adhesive bandage of claim 1, wherein each of plurality of flexion sites includes a concave curvature at an inward apex of each of plurality of flexion sites.

12. The conformable adhesive bandage of claim 1, further comprising an absorbent pad disposed centrally on the central body on the first side of the conformable adhesive bandage.

13. The conformable adhesive bandage of claim 1, further comprising at least one removable backing disposed on the first side of the conformable adhesive bandage.

14. The conformable adhesive bandage of claim 1, wherein the plurality of flexion sites includes a plurality of central body flexion sites, the plurality of central body flexion sites being disposed at a plurality of vertices along the central body between adjacent bandage limbs of the plurality of bandage limbs, and the plurality of central body flexion sites inflect radially inward at the plurality of vertices into the central body.

15. The conformable adhesive bandage of claim 1, wherein the plurality of flexion sites includes a plurality of bandage limb flexion sites disposed along at least one of the plurality of bandage limbs and inflecting inward into the at least one of the plurality of bandage limbs.

16. The conformable adhesive bandage of claim 15, wherein the plurality of flexion sites includes the plurality of bandage limb flexion sites disposed along each of the plurality of bandage limbs and inflecting inward into each of the plurality of bandage limbs

17. The conformable adhesive bandage of claim 15, wherein the plurality of bandage limb flexion sites is divided into pairs, each bandage limb flexion site of a pair of bandage limb flexion sites being disposed in opposing edges of a bandage limb at an equivalent position along a length of the bandage limb.

18. The conformable adhesive bandage of claim 1, wherein at least one of the plurality of bandage limbs includes a first bandage limb length, and at least one of the plurality of bandage limbs includes a second bandage limb length, the second bandage limb length being longer than the first bandage limb length.

19. A conformable adhesive bandage, comprising:
a central body;
a plurality of bandage limbs including an adhesive coating disposed on a first side of the conformable adhesive bandage; and
a plurality of flexion sites, the plurality of flexion sites being disposed at a plurality of vertices along the central body between adjacent bandage limbs of the plurality of bandage limbs,
wherein the plurality of flexion sites inflect radially inward at the plurality of vertices into the central body.

20. A conformable adhesive bandage, comprising:
a central body;
a plurality of bandage limbs including an adhesive coating disposed on a first side of the conformable adhesive bandage;
a plurality of central body flexion sites, the plurality of central body flexion sites being disposed at a plurality of vertices along the central body between adjacent bandage limbs of the plurality of bandage limbs, and the plurality of flexion sites inflect radially inward at the plurality of vertices into the central body; and
a plurality of bandage limb flexion sites disposed along at least one of the plurality of bandage limbs and inflecting inward into the at least one of the plurality of bandage limbs, the plurality of bandage limb flexion sites being divided into pairs and each bandage limb flexion site of a pair of bandage limb flexion sites being disposed in opposing edges of a bandage limb at an equivalent position along a length of the bandage limb.
